(19) United States
(12) Patent Application Publication
(10) Pub. No.: US 2011/0311588 A1
(43) Pub. Date: Dec. 22, 2011

(54) AGENTS AND TREATMENT FOR SNORING AND RESPIRATORY EFFORT RELATED AROUSALS IN SLEEP

(76) Inventor: Phillip Comans, New South Wales (AU)

(21) Appl. No.: 13/129,034

(22) PCT Filed: Nov. 9, 2009
(86) PCT No.: PCT/AU09/01451

§ 371 Date: Jul. 19, 2011

(30) Foreign Application Priority Data
Nov. 13, 2008 (AU) 2008905902

(51) Int. Cl. A61K 39/085; A61P 37/04; A61P 11/02; A61P 31/04

(52) U.S. Cl. 424/243.1

(57) ABSTRACT

A method for prophylaxis or treatment of respiratory effort related arousals during sleep of a subject is provided. The method comprises administering to the subject an effective amount of an agent for reducing or inhibiting infection or colonisation of the nasal cavity of the subject by Staphylococcus aureus. Typically, the respiratory effort related arousal is associated with snoring. The agent may be selected from the group consisting of antibiotics, immunostimulants, probiotics and mixtures of the foregoing. A method for prophylaxis or treatment of snoring comprising administration of the agent to the subject is also provided.

### FIELD OF THE INVENTION

The invention relates to a method for prophylaxis or treatment of sleep related conditions in a subject using an agent for reducing or inhibiting colonisation of Staphylococcus aureus of the nasal cavity of the subject.

### BACKGROUND OF THE INVENTION

Snoring arises from oscillation of the oropharangeal soft tissues and is a widespread phenomenon amongst the general population with some reports suggesting that at least 20% of people snore. It is most common in middle aged males although women become more prone to snoring after menopause. Snoring has also been associated with adverse health outcomes including fatigue and hypertension, and the more serious conditions of ischemic heart disease and brain ischemia.

Research into snoring indicates that it commonly has more than one cause and a number of risk factors have been identified including smoking, alcohol, obesity, physiological causes such as deviated nasal septum and other abnormalities in internal nasal structures, chronic nasal congestion, congestion of throat tissues, lack of fitness and muscle tone, hypertrophy of the tonsils and/or adenoids and tongue enlargement. Medications such as benzodazepines and tranquilisers can also result in muscle relaxation thereby increasing the risk and/or tendency for snoring. Snoring can be divided into a number of categories ranging from mild snoring to severe snoring involving hypopnea during which a person's breathing may become abnormally slow and shallow, and apneic events during which breathing stops. The latter of these is characteristic of obstructive sleep apnea (OSP) involving complete airway closure.

Chronic nasal congestion at night has been reported as a risk factor for habitual snoring, including snoring without frank sleep apnea (Young et al, 2001). Congestion due to allergies was not found to be a stronger predictor of snoring than other causes such as deviated septum. Various treatments are available for snoring ranging from the use of decongestants and adhesive nasal strips which are adhesively fastened to the bridge and the tip of the nose to open the nasal passages, to masks connected to air pumps that act to apply a continuous positive air pressure to maintain the breathing passage open. However, the use of interventions such as adhesive nasal strips can be uncomfortable and may cause skin allergies and irritations, while apparatus for applying a continuous positive air pressure to alleviate snoring is inconvenient and can be cumbersome and expensive. Air pumps used in such equipment can also be relatively noisy thereby disrupting the sleep of sleeping partners and others in hearing range of the pump.

The ingestion of a probiotic milk drink containing Lactobacillus GG, Streptococcus thermophilus, Lactobacillus acidophilus and Bifidobacterium sp. has been reported to reduce nasal colonisation of the potentially pathogenic bacteria (PPB) Staphylococcus aureus, Streptococcus pneumoniae and -hemolytic streptococci (Glück and Gebbers, 2003). Antiseptic regimens had been suggested as potentially being crucial for infection control of patients carrying PPB after major operations on, or injuries to, the head, nasal sinuses, or lungs, and possibly also for diabetic patients and persons receiving haemodialysis, in intensive care units, or with impaired immunity, and that study was undertaken to test the possible effect of the ingestion of probiotics on the bacterial flora of the nose.

### SUMMARY OF THE INVENTION

Staphylococcus aureus is a bacterial pathogen commonly found on the skin and in the nasal cavity (e.g., the nares), and can cause pus-forming infections such as pimples, styes, boils, impetigo and abscesses in the skin as well as serious infections in the bloodstream and joints including meningitis, lung pneumonia, endocarditis (infection of heart valves) and septic phlebitis if it enters the body through cuts, abrasions or wounds. Localised host responses to S. aureus can involve inflammation of tissues. Several strains of antibiotic resistant S. aureus (e.g., methicillin resistant) are known and cause significant infection problems in hospitals where the pathogen can enter the body via catheters or, for instance, during dialysis or surgery. In the broader community, S. aureus strains are frequently susceptible to a range of commonly used antibiotics. Nevertheless, patients, hospital workers and persons in the general public can become colonised by the pathogen and serve as a reservoir for transmission of the pathogen. It has been suggested that up to 2 out of every 10 persons may be colonised by the pathogen.

The invention stems from the recognition that S. aureus infection or colonisation of the nasal cavity may impact on breathing during sleep and be associated with snoring. While snoring has detrimental effects such as fatigue and drowsiness in the sufferer, not all episodic upper airway resistance causes snoring (which may be multifactorial in nature) but nevertheless, can result in brief arousals from sleep. This phenomenon is known as “respiratory effort related arousals”, which can lead to more serious sleep disorders and/or sleep related breathing disorders, and may also cause fatigue and drowsiness.

Broadly stated, the invention relates to the administration of an agent for reducing or inhibiting infection or colonisation of the nasal cavity of the subject by Staphylococcus aureus to alleviate respiratory effort related arousals during sleep and/or improve the quality of sleep in the subject.

Thus, in one aspect of the invention there is provided a method for prophylaxis or treatment of respiratory effort related arousals during sleep of a subject, comprising administering to the subject an effective amount of an agent for reducing or inhibiting infection or colonisation of the nasal cavity of the subject by S. aureus.

The respiratory related sleep arousals may or may not be associated with snoring. Nevertheless, embodiments of the invention have particular application to prophylaxis or treatment of snoring.

Hence, in another aspect of the invention there is provided a method for prophylaxis or treatment of snoring by a subject, comprising administering to the subject an effective amount of an agent for reducing or inhibiting infection or colonisation of the nasal cavity of the subject by S. aureus.

Typically, the agent will inhibit infection or colonisation by S. aureus of the nares of the nasal passage.

The agent may be any therapeutic agent that can directly or indirectly reduce or effect the inhibition of S. aureus infection or colonisation in the nasal passage and can, for example, be selected from the group consisting of antibiotics and immunostimulants. The term “immunostimulant” refers to an agent that stimulates a specific or non-specific immune response in the subject against S. aureus and includes probiotics.

The probiotic can be a whole (viable/alive or killed) cell or part thereof for stimulating the immune system against S. aureus. Typically, the probiotic will stimulate the common immune system of the subject and stimulate a Th1 immune response and/or suppress a Th2 immune response in the subject.

Moreover, in at least some embodiments, the immunostimulant can be a preparation of S. aureus antigen for generating a specific immune response against infection or colonisation of the nasal cavity by the pathogen.

In another aspect of the invention there is provided a method for improving sleep quality of a subject, comprising administering to the subject an effective amount of an agent for reducing or inhibiting infection or colonisation of the nasal cavity of the subject by S. aureus.

In another aspect of the invention there is provided the use of an agent for reducing or inhibiting infection or colonisation of the nasal cavity of a subject by S. aureus for prophylaxis or treatment of respiratory effort related arousals during a sleep of the subject.

In another aspect of the invention there is provided the use of an agent for reducing or inhibiting infection or colonisation of the nasal cavity of a subject by S. aureus for improving quality of sleep in the subject.

In another aspect of the invention there is provided the use of an agent for reducing or inhibiting infection or colonisation of the nasal cavity of a subject by S. aureus for prophylaxis or treatment of snoring by the subject.

The subject can be any mammalian animal model for snoring or a human being. Typically, the subject will be a human.

All publications mentioned in this specification are herein incorporated by reference. Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed in Australia or other jurisdictions before the priority date of this application.

The features and advantages of the invention will become further apparent from the following detailed description of embodiments thereof together with the accompanying drawings.

### BRIEF DESCRIPTION OF THE ACCOMPANYING DRAWINGS

FIG. 1 shows changes in a male chronic carrier of s. aureus in relation to snoring and in associated factors of sleep disorder after a single dose of Mupirocine ointment was administered to the nostril regions of the subject. There was an initial dramatic effect on each parameter recorded which lasted about 3 days. Symptoms then began to return but had not returned to baseline levels after 2 weeks; and

FIG. 2 shows the changes in nasal blockage in the male chronic carrier of S. aureus in snoring and in associated factors of sleep disorder during daily treatment with probiotic (Lactobacillus acidophilus/Bifidusbacterium animalis). There was an initial dramatic effect on each parameter recorded which lasted about 3 days and then stabilised during treatment period. The spike at day 8 was associated with particularly stressful situation reported by the subject.

### DETAILED DESCRIPTION OF EMBODIMENTS OF THE INVENTION

The invention extends to the use of antibiotics and immunostimulants (e.g., probiotics and S. aureus antigen preparations) to reduce or inhibit colonisation of the nasal passage by S. aureus for prophylaxis or treatment of snoring.

Any of a range of antibiotics can be employed in a method embodied by the invention. Many strains of S. aureus are now resistant to penicillin primarily because the bacteria produce the enzyme -lactamase which degrades penicillin destroying its antibacterial activity. Although penicillin can still be used to treat S. aureus strains responsive to it, other antibiotics will generally be employed including, but not limited to, methicillin, flucloxacillin, mupirocin (e.g., Bactroban™ nasal), erythromycin, ciprofloxacin (e.g., Ciproxin™), vancomycin and teicoplanin (e.g., Targocid™). Methicillin resistant strains of S. aureus are known as MRSA. MRSA may still be treated with mupirocin or, for example, vancomycin or teicoplanin. The selected antibiotic can be administered orally, intravenously or to mucosa of the nasal cavity. In at least some embodiments the antibiotic is applied topically to the nares of the subject in a topically acceptable carrier (e.g., as a cream or ointment). Topical application of the antibiotic is preferred as extended oral use of antibiotics can impact on intestinal bacterial flora populations and have unintended side effects including diarrhoea.

The term “probiotic” encompasses bacteria and yeast, and parts thereof (e.g., fraction(s) or specific cell surface components of the probiotic) which act to reduce or inhibit infection or colonisation of the nasal cavity by S. aureus. The probiotic can be a killed or viable (live) whole cell. Typically, the probiotic will be viable whole cells.

Particularly suitable bacterial probiotics include strains of the genera Lactobacillus, Bifidobacterim, Brevibacterium, Propionibacterium and Mycobacterium. Yeast probiotics that may have application in embodiments of the invention include strains of the genera Sacchromyces such as Sacchromyces boulardii. Examples of bacterial probiotics include, L. acidophilus, L. fermentum, L. casei, L. plantarum and L. rhamnosus, M. vaccae, B. breve, P. jensenii. The probiotic can be taken as a food (which can be a fermented or non-fermented food) or as a probiotic supplement preparation such as a concentrated probiotic drink or in capsule form or in tablet form. Probiotics in capsule form are widely commercially available and are particularly suitable for use in methods as described herein. Likewise, tablets containing whole killed probiotic cells (e.g., heat killed) or parts thereof can be taken orally. The capsules and tablets will may be enterically coated for passage of the probiotic through the acid environment of the stomach and release in the small intestine. The capsule or tablet may also contain prebiotic as well as probiotic. The prebiotic can, for example, be selected from carbohydrates, monosaccharides and oligosaccharides although non-carbohydrate prebiotics are not precluded. Examples of prebiotic monosaccharide and oligosaccharides that may be utilised include fructooligosaccharides (FOS), xylooligosaccharides (XOS), polydextrose, galactooligosaccharides (GOS) and the monosaccharide tagatose.

Orally administered probiotics can be taken up by lymphoid nodes known as Peyer's patches in the small intestine where they are processed and probiotic antigen is presented (i.e., by dendritic cells and macrophages) to effector immune cells. The effector cells migrate via efferent lymphatics to distant mucosal sites including respiratory and nasal cavity mucosal surfaces where they can exert a non-specific cellular-mediated immune response providing mucosal protection against S. aureus infection or colonisation. Thus, administration of probiotic(s) via the oral route can stimulate an immune response at remote mucosal surfaces, this system being known as the “common mucosal system” (see for example International Patent Application No. PCT/AU01/00726).

Effector T lympocytes are responsible for the cell-mediated immune responses of adaptive immunity and may be broadly categorised into three groups namely, cytotoxic T cells, Th1 cells and Th2 T-cells. Th1 cells stimulate antibacterial mechanisms of phagocytic cells such as neutrophils and macrophages, and release cytokines that attract phagocytic cells to the site of infection/colonisation. Th2 cells have a role in activating B-cells for generating antibodies against bacterial and other antigens. The probiotic(s) or part(s) thereof utilised in a method of the invention will typically generate a Th1 immune response and/or down-regulate/suppress Th2 responses. Similarly, any adjuvant(s) added to the immunostimulant will also typically be selected to generate a Th1 immune response.

Cytokines typically secreted by Th1 cells include γ-interferon (γ-IFN), IL-12 and TNF-β. γ-IFN is the main phagocytic cell activating cytokine. TNF-β is directly cytotoxic for some cells. In contrast, Th2 cells secrete IL-4, IL-5, IL-10, IL-13, TGF-β and other cytokines. While both Th1 and Th2 cells both secrete IL-3, GM-CSF and for instance TNF-α, the overall cytokine profiles of each type of cell are different. Hence, a Th1 response can be detected by up-regulated secretion of a cytokine or combination of cytokines characteristic of a Th1 immune response such as γ-IFN and/or IL-12. Similarly, a Th2 immune response may be characterised by up-regulated expression of a cytokine or combination of cytokines characteristic of a Th2 response such as IL-4 or IL-10.

Immunostimulants that can be employed in methods embodied by the invention include preparations of S. aureus antigen (e.g., attenuated, whole killed or particulate antigen) which can generate a specific and/or systemic immune response against the pathogen. While it is desirable that the immune response be specific against S. aureus it is not essential, and antigen from other bacteria or sources that generate a non-specific immune response that reduces or inhibits S. aureus infection or colonisation of the nasal cavity can also be used. The immunostimulant may include an adjuvant such as may be selected from, for instance, cholera toxin B subunits and conventionally known alum adjuvants.

Particularly suitable immunostimulants include immunizing preparations of S. aureus clumping factor B (ClfB) which has been shown to reduce nasal colonisation of S. aureus in a murine model (Schaffer et al., 2006). In this study, S. aureus mutants that lack clumping factor A (ClfA), collagen binding protein, fibronectin binding proteins A and B, polysaccharide intracellular adhesion, or the accessory gene regulator, colonized as well as wild-type strains of the pathogen. However, mutants deficient in ClfB or sortase A showed reduced colonisation. Moreover, mice immunised systemically or intranasally with a recombinant vaccine composed of domain A of ClfB were found to exhibit reduced nasal colonisation by S. aureus, as were mice passively immunised with a monoclonal antibody (MAb) against ClfB. ClfB is abundantly expressed in the log growth phase when capsule is not present but not in the stationary phase when capsule is present. Hence, S. aureus expressing ClfB for use as antigen as described herein should be harvested in the log growth phase when ClfB is expressed.

The nose of the mouse is known to contain a high density of immune nodes equivalent to the Peyer's patches in humans. Such nodes are also found in the nasal passageway of humans, and administration of S. aureus antigen as described above intranasally (e.g., by spray) or orally offers a mechanism for stimulating an immune response against the pathogen.

Cellular fractions of probiotics and S. aureus can also be employed in methods embodied by the invention. The fraction can be prepared by disrupting killed or viable microorganism(s), and filtering the resulting product to obtain cellular material within a discrete size range. Any suitable method which achieves an appropriate level of cellular disruption can be employed including dissolution of cells utilising appropriate surfactants and agitation. Generally, the microorganism(s) will be subjected to sonication. Without being limited by theory, probiotic cellular debris may include natural adjuvant(s) which helps stimulate a mucosal immune response against S. aureus. The natural adjuvant(s) may for example comprise lipopolysaccaride and CpG oligodeoxynucleotides. The sonication step can be repeated a number of times in order to obtain the desired degree of disruption of the microorganism and the release or generation of appropriate sized soluble antigen/cellular material. The number of cycles and length of each may be determined by repeating the process a number of times employing a different number of cycles each time. Alternatively, or as well, the length of time the microrganism is sonicated may be varied. The collected fraction can then be tested for ability to produce an immune response against S. aureus.

An immunostimulant as described herein will comprise sufficient antigen such that an effective dosage will be delivered to the subject for the generation of the immune response taking into account the proposed mode of delivery and added adjuvant(s) (if any) as can be determined using well accepted principles in the art of immunisation. Typically, the dosage of an immunostimulant comprising S. aureus antigen will typically be in a range of about 10⁹ to about 10¹² viable or killed bacteria, and more usually in a range of from about 10¹⁰ to about 10¹¹ viable or killed bacteria. The optimum dosage of the antigen may be determined by administering different dosages to different groups of test mammals, prior to subsequently intransally infecting the animals in each group with S. aureus, and determining the dosage level required to achieve satisfactory clearance of the pathogen or inhibition of infection/colonisation. Moreover, the immunostimulant can be administered in accordance with any regimen suitable for generating an effective immune response against S. aureus infection or colonisation of the nasal cavity. For example, a single dose of the immunostimulant can be administered. One or more “booster” doses administered at an interval of a number of weeks or months may also be given.

As will be understood by the skilled addressee, any suitable formulations, preparations, delivery forms and/or compositions suitable for the delivery, consumption or administration of the agent for reducing or inhibiting the infection or colonisation of S. aureus in the nasal cavity can be utilized. For instance, as described above, a probiotic can be taken in a concentrated capsule form or tablet form. Typically, whole live probiotic in a range of from about 2×10⁹ to 3×10¹¹ cells will be taken be orally by the subject on a daily basis for about a 1 day to 2 weeks to stimulate the non-specific mucosal immune response against S. aureus. The subject can then be administered an ongoing daily maintenance dosage of 1×10⁹ to 1×10¹¹ probiotic cells. Alternatively, probiotics can also be consumed as a fermented or non-fermented food, the food being consumed over a sufficient period (e.g., weeks) for generation of a non-specific mucosal immune response to reduce or inhibit infection or colonisation of S. aureus in the nasal cavity. Fermented probiotic food contains live probiotic micro-organisms which have grown in the food and produced a fermented product. Non-fermented probiotic food contains probiotic micro-organisms or their components which have been added to the food. Fermented probiotic foods include probiotic dairy foods such as yoghurt including drinking yoghurt, and fermented milks. The invention also extends to the use of mixed cultures of two or more probiotic organisms and includes cultures of lactobacillus with one or more of bifidobacterium, brevibacterium, and/or propionibacterium strains.

Probiotic and immunostimulant preparations as described herein can also contain one or more anti-caking agents and preservatives (e.g., thimerosal) suitable for the proposed mode of administration, stabilisers such as amino acids and/or sugar moieties, sweetening agents such sucrose, lactose or saccharin, surfactants, pH buffering agents and pH modifiers such as monosodium phosphate and/or disodium phosphate, a pharmaceutically acceptable carrier such as physiologically saline, solvents and dispersion media. Use of such ingredients and media in pharmaceutical preparations is well known in the art. Except insofar as any conventional media or agent is incompatible with the S. aureus isolate(s) or antigens, or the proposed mode of administration, their use is specifically encompassed. Formulations and suitable pharmaceutically (including topically) acceptable carriers useful in the present invention can be found in handbooks and texts well known to the skilled addressee such as “Remington: The Science and Practice of Pharmacy (Mack Publishing Co., 1995)”, the contents of which is incorporated herein in its entirety by reference.

The invention is described further below by way of a number of non-limiting Examples. For the purpose of treating individuals, live probiotic bacteria were used unless otherwise indicated.

### Example 1

### Effect of Antibiotic Ointment on Snoring and Sleep Quality

A study was conducted to assess the effects of Mupirocine ointment on snoring and sleep pattern in a male subject with a history of snoring and sleep disturbance. The subject had been identified as having nares chronically colonised with Staphylococcus aureus (methicillin sensitive S. aureus) prior to the commencement of the study.

At bedtime in the evening of the commencement of the study, Mupirocine ointment (Bactroban™, GlaxoSmithKline) was applied at the base of each nostril of the subject as well as just inside his nostrils. The subject's sleeping partner (wife, aged 46 years) was unwilling to not use ear plugs that night. Follow up observations were recorded over the subsequent 16 day period, and the degree of nasal blockage and snoring of the subject as well as morning freshness of he and his wife was assessed. Mupirocine ointment was not administered to the subject during the 16 day observation period.

### 1. Demographic Details of the Subject

Details for the subject including medical history are shown in Tables 1-3 below.

TABLE 1
Personal details of subject

| | |
|---|---|
| Sex | male |
| Date of birth | 29 Jul. 1957 |
| Age (years) | 51 |
| Weight (kg) | 82 |
| Height (cm) | |
| Smoking history | Never smoked |

TABLE 2
Medical history of subject

| Disease/condition | Started | Ongoing/date finished |
|---|---|---|
| Coronary artery bypass graft | 19 Dec. 2007 | na |
| Hypertension | 2004 | ongoing |
| Coronary artery disease | 2004 | ongoing |
| Hypercholesterolemia | 2004 | ongoing |
| Angina pectoris | September 2007 | December 2007 |
| Mild depression | 2002 | ongoing |
| Mild knee arthritis | 1990 | ongoing |

TABLE 3
Concomitant medication taken by subject

| | | | Was it |
|---|---|---|---|
| | | | taken |
| | | Dose form & | on the |
| Medication | Reason taken | dose | study day? |
| Cipramil | Mild depression | Oral, 20 mg od | yes |
| Ramipril | Hypertension | Oral, 2.5 mg od | yes |
| Aspirin | Coronary artery | Oral, 100 mg od | yes |
| | disease | | |
| Ezitamibe | Cholesterol | Oral, 100 mg od | yes |
| Nicotinic acid | Triglycerides | Oral, 12.5 mg bid | yes |
| Blackmore's | General health | Oral, od | yes |
| Slow Release | | | |
| Mulitvitamin | | | |
| Blackmore's | Arthritis | Oral, bid | yes |
| Joint formula | | | |
| (Glucosamine250 | | | |
| mg/Chondroitin 750 mg) | | | |

### 2. Sleep History of Subject

Previous tests in clinical and experimental settings have shown that the nares of the subject's nose have been chronically blocked. For the week prior to the commencement of the study, the subject had blocked nares with feelings of heaviness in the face, day-time tiredness, night-time snoring and disturbed sleep, and had awoken sleep deprived. The subject's wife (aged 46 years) had been disturbed by the snoring despite wearing ear plugs forcing her to sleep in a different room to the subject on some nights. On one occasion, the subject's son shut both his own bedroom door as well as the subject's bedroom door in order to avoid his own sleep being disturbed by the subject's snoring.

### 3. Results

The subject awoke feeling refreshed with clear nares in the early hours of the morning following the administration of the Mupirocine ointment. Both the subject and his wife reported that he did not snore during the night.

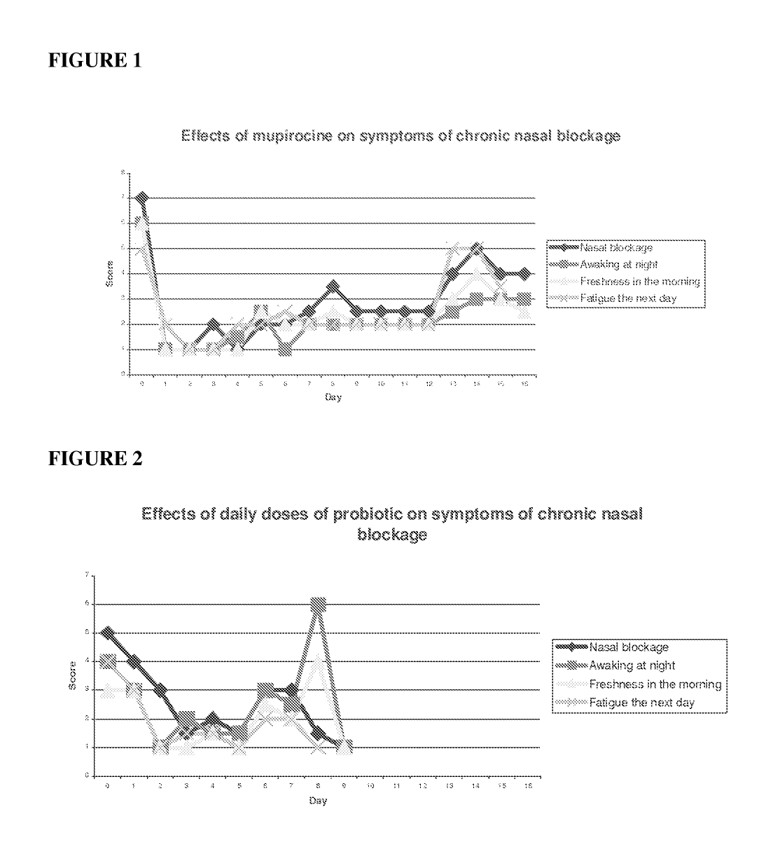

Follow-up daily observations over the next 16 days showed that the nares of the subject remained clear for at least 3 days (see FIG. 1). While the subject reported the sensation of nasal blockage and symptoms of sleep disturbance returned during the 16 day period, they still showed an approx. 50% improvement when compared with baseline. The subject was not treated further with the Mupirocine ointment during this period. Corresponding daily observations were also made by the subject's wife for a period of 7 days following the treatment of the subject with the Mupirocine ointment.

Self assessment parameters recorded by the subject and his wife following the treatment of the subject with Mupirocine ointment are shown in Table 4 and Table 5. The results were scored on a scale of 1 to 7, with 1 being very good and 7 being very poor. The subject's daily scores are graphed in FIG. 1.

TABLE 4
Subject - self assessment

| | Previous nights (without | |
|---|---|---|
| Symptom | Mupirocine) | With Mupirocine |
| Nasal blockage | 7 | 1 |
| Awaking during the night | 6 | 1 |
| Freshness in the morning | 6 | 1 |

TABLE 5
Subject's wife - self assessment

| | Previous nights (without | |
|---|---|---|
| Symptom | Mupirocine) | With Mupirocine |
| Wife's awaking during the | 6 | 2 |
| night | | |
| Wife's awaking at night due | 6 | 1 |
| to subject's snoring | | |
| Loudness of subject's snoring | 6 | 1 |
| Freshness in the morning | 6 | 4 |

As indicated in Table 5, prior to the treatment of the subject with the Mupirocine ointment, the subject's wife recorded scores of 6 for all parameters measured despite her wearing ear plugs. The subject's wife woke about 5.00 am due the subject's “shuffling” in bed which impacted on her freshness in the morning. She was not aware of any snoring by the subject during the night.

During the subsequent 7 days, the averages of the scores recorded by the subject's wife showed that all parameters evaluated had improved, with substantial reductions in both the loudness of the subject's snoring and being awakened at night by the subject's snoring being recorded.

### 4. Discussion

The results indicate that reducing colonisation of S. aureus in the nasal passage by treatment with Mupirocine (antibiotic) ointment reduces snoring and improves sleep quality for both the snorer and sleeping partner. Further, the improvement in sleep quality reduces fatigue in both the snorer and sleeping partner the following morning.

### Example 2

### Effects of Probiotics L. acidophilus/Bifidus animalis on Snoring and Sleep Quality

The subject (male) described in Example 1 has a history of snoring, sleep disturbance and daytime fatigue. This contributes to sleep disturbance of his sleeping partner (wife). As outlined in Example 1, investigations have shown that the subject's nares are chronically colonised with Staphylococcus aureus (methicillin sensitive S. aureus). Probiotics (L. acidophilus/Bifidus animalis) are known to generally enhance immune system performance via the common mucosal immune system. A study was conducted to determine whether oral treatment of the male subject with probiotic (L. acidophilus/Bifidus animalis) would reduce nasal colonisation by S. aureus and improve the snoring and sleep patterns of the subject.

The subject was given an initial dose of 2 capsules containing the probiotic orally 3 times daily for 9 days (white capsules containing Lactobacillus acidophilus/bifidus animalis, 10⁹ cells/capsule; Blackmore's, Warriewood, NSW, Australia)), then a maintenance dose of the probiotic of 1 capsule, 3 times daily for 14 days. Observations were recorded daily during the study period, and parameters including the degree of nasal blockage and snoring of the subject as well as morning freshness of he and his wife was self assessed. Observations were also recorded on the day (Day 0) prior to commencement of the study. Self assessment parameters recorded by the subject were scored on a scale of 1 to 7, with 1 being very good and 7 being very poor. The subject's results are presented below.

### 1. Results

There were statistical differences between pre-treatment and probiotic in relation to nasal block, evidence of S. aureus colonisation, whether measured by t-test (p=0.0027) or Confidence intervals. There were also statistical differences between post-treatment and probiotic in relation to nasal block whether measured by t-test (p=0.0002) or Confidence intervals.

An Intent to treat analysis (ITT) was also performed between treatment with probiotic and pre-treatment scores. When single outlier scores for waking at night and morning freshness, which occurred during a time of particular stress, were deleted, t-tests showed statistical differences between pre-treatment and treatment. When measured using Confidence intervals (not ITT), there were differences between pre-treatment and each of the treatment test scores. Results for the Intent to treat analysis are shown in Table 6.

TABLE 6
Intent to treat analysis results

| | | | | | Waking at | | Morning | | | |
|---|---|---|---|---|---|---|---|---|---|---|
| | Nasal block | | Nasal drip | | night | | freshness | | Fatigue | |
| | Pre | Tx | Pre | Tx | Pre | Tx | Pre | Tx | Pre | Tx |
| Mean | 5.0 | 2.3 | 5.0 | 1.6 | 4.0 | 2.2 | 3.0 | 1.7 | 4.0 | 1.6 |
| SD | 0 | 1.01 | 0 | 1.15 | 0 | 1.42 | 0 | 0.93 | 0 | 0.59 |
| CI +/− | 5 | 2.8/1.7 | 5 | 2.2/1.0 | 4 | 2.9/1.4 | 3 | 2.2/1.3 | 4 | 1.9/1.3 |

| | | | | | |
|---|---|---|---|---|---|
| p | 0.0027 | 0.0013 | 0.1071 | 0.0962 | 0.0001 |

As can be seen from Table 6, statistical differences (ITT analysis) between treatment with probiotic and post-treatment scores were found for:
reduction in nasal blockage (p<0.001)
night time wakefulness (p=0.02)
freshness in the morning (p=0.01)
day-time fatigue (p<0.001)

The differences between probiotic and post-treatment scores were consistent whether measured by t-test or Confidence intervals.

Benefit occurred on the first day of treatment although maximum benefit occurred by day 3. The benefits lasted while treatment was maintained (14 days) after which there was no statistical difference between post-treatment and pre-treatment scores.

The subject reported reduced nasal blockage and improved sleep patterns during the course of the study and his daily scores for the initial 9 days of the study period are shown graphed in FIG. 2. In each parameter assessed there was an initial dramatic effect which lasted about 3 days, which then stabilised during the treatment period. The spike shown at day 8 in FIG. 2 was associated with a particularly stressful event reported by the subject. The subject's wife also reported substantial reductions in the snoring of the subject as well as the loudness of the snoring.

### 2. Discussion

The study shows that daily probiotic (acidophilus/bifidus) reduces snoring and improves sleep quality Improvement in sleep quality improves morning freshness and reduces daytime fatigue. Probiotic acts by mildly boosting the immune system. Other methods to reduce specifically boost the immune system targeted specifically at nasal colonisation of S. aureus would also be expected to improve sleep quality.

### Example 3

### Dose-Response Effects of Probiotics L. acidophilus/Bifidus animalis on Snoring and Sleep Quality

The subject (male) described in Examples 1 and 2 was assessed in a third study for dose-response to probiotic treatment, to determine whether oral treatment of the subject with probiotic (L. acidophilus/Bifidus animalis) doses of 2×10⁹ and 5×10⁹ bacteria reduced nasal colonisation by S. aureus and improved the snoring and sleep patterns of the subject.

After a 9 day wash-out period, the subject was given an initial dose of 5 capsules containing the probiotic orally (white capsules containing Lactobacillus acidophilus/bifidus animalis, 10⁹ organisms/capsule; Blackmore's, Warriewood, New South Wales, Australia) for 4 days then 2 capsules for 3 days. Self assessment parameters recorded by the subject and his wife were scored on a scale of 1 to 7, with 1 being very good and 7 being very poor.

### 1. Results

There were statistical differences between pre-treatment and probiotic in relation to nasal block, evidence of S. aureus colonisation, whether measured by t-test (p<0.001) or Confidence intervals. There were also statistical differences between a dose of 5 capsules 3 times daily and a dose of 2 capsules 3 times daily for both nasal blockage and fatigue the next day (p<0.05).

TABLE 7
Intent to treat analysis

| | | | | p-values | |
|---|---|---|---|---|---|
| | Mean | | | 2 x vs | |
| | Run-in | 5 x | 2 x | Run-in | 5 x vs 2 x |
| Nasal block | 4.6 | 1.2 | 2.5 | <0.05 | <0.05 |
| Nasal drip | 2.9 | 1.1 | 1.5 | <0.05 | 0.11 |
| Waking at night | 3.3 | 2.0 | 2.2 | 0.07 | 0.50 |
| Morning freshness | 3.2 | 1.2 | 2.0 | 0.10 | 0.11 |
| Daytime fatigue | 3.4 | 1.5 | 2.5 | <0.05 | <0.05 |

With a dose of 5 capsules, 3 times daily, the initial peak benefit was achieved within 1 hour compared with 3 days in Example 2 (dose 1 capsule, 3 times daily). With a dose of 5 capsules, the benefit lasted for up to 8 hours after the final daily dose compared with 5-6 hours with a dose of 2 capsules.

The subject reported a dose-response effect in relation to nasal blockage with S. aureus and each symptom of snoring.

### 2. Discussion

The study shows that daily probiotic (acidophilus/bifidus) reduces snoring and improves sleep quality, and that there is a dose-dependent effect on effectiveness, time to onset of effect and duration of effect.

### Example 4

### Individual Subject Observation I

A 58 year old female had suffered from chronic congestion in the left hand side of the face for more than one year. The individual uses CPAP (continuous positive airways pressure) for snoring, has chronic and nasal congestion. The nasal congestion causes discomfort and pain. The individual was treated with 1× probiotic (L. acidophilus/B. Animalis; 2.5×10¹⁰ bacteria) every evening before going to bed.

### 1. Results

After four days the congestion eased, and after one week it was gone. Soundness of sleep improved while continuing to use the CPAP. The individual switched to taking the probiotic in the morning, and after 6 weeks had not experienced congestion at all during that time.

### 2. Discussion

The study shows that the daily administration of probiotic (acidophilus/bifidus) reduces snoring and improves sleep quality

### Example 5

### Individual Subject Observation II

A 52 year old male had suffered from chronic congestion, snoring and sleep disturbance. The individual was treated with 1× probiotic (L. acidophilus/B. Animalis; 1−2.5×10¹⁰ bacteria) every evening before going to bed.

### 1. Results

After six months of treatment, sleep was consistently improved. There was noticeable worsening of sleep when probiotic was missed several nights in succession. The individual's wife reported that while snoring sometimes occurred, it was substantially less loud, less frequent and less disturbing of her night's sleep when the probiotic was used.

### 2. Discussion

The study shows that daily administration of probiotic (acidophilus/bifidus) reduces snoring and improves sleep quality.

### Example 6

### Individual Subject Observation III

The 52 year old male described in Example 5 had suffered from chronic congestion, snoring and sleep disturbance and had been maintained on capsules containing L. acidophilus/B. Animalis. The individual was treated with 1× probiotic (L. fermentum; >1×10⁹ bacteria) every evening before going to bed.

### 1. Results

After 1 week of treatment with L. fermentum the improvement in sleep was maintained. However, surprisingly, the individual reported L. fermentum provided better sensation/less irritation in the nasal cavities.

### 2. Discussion

The study shows the daily administration of L. fermentum reduces snoring and improves sleep quality.

Although the invention has been described with reference to a number of embodiments, it will be apparent to those skilled in the art that numerous variations and/or modifications can be made. The present embodiments are, therefore, to be considered in all respects as illustrative and not restrictive.

### REFERENCES

Glück, U., and Gebbers, J-O., Ingested probiotics reduce nasal colonization with pathogenic bacteria (Staphylococcus aureus, Streptococcus pneumoniae, and -hemolytic streptococci). Am J Clin Nutr., (2003), Vol. 77, pp. 517-20.
Schaffer, A. C., et al, Immunization with staphylococcus aureus clumping factor B, a major determinant in nasal carriage, reduces nasal carriage in a murine model. Infection and Immunity, (2006), Vol. 74, pp. 2145-2153.
Young, T., et al, Chronic nasal congestion at night is a risk factor for snoring in a population-based cohort study. Arch Intern Med., (2001), Vol. 161, pp. 1514-1519.

1. A method for prophylaxis or treatment of respiratory effort related arousals during sleep of a subject, comprising administering to the subject an effective amount of an agent for reducing or inhibiting infection or colonisation of the nasal cavity of the subject by Staphylococcus aureus.

2. A method according to claim 1 wherein the respiratory effort related arousals are associated with snoring.

3. A method for prophylaxis or treatment of snoring by a subject, comprising administering to the subject an effective amount of an agent for reducing or inhibiting infection or colonisation of the nasal cavity of the subject by Staphylococcus aureus.

4. A method according to claim any one of claims 1 to 3 wherein the agent inhibits infection or colonisation by S. aureus of the nares of the nasal passage.

5. A method according to any one of claims 1 to 4 wherein the agent is selected from the group consisting of antibiotics and immunostimulants.

6. A method according to claim 5 wherein the agent is an antibiotic.

7. A method according to claim 5 comprising administering an immunostimulant to the subject.

8. A method according to claim 7 wherein the immunostimulant stimulates a specific immune response against S. aureus.

9. A method according to claim 7 or 8 wherein the immunostimulant comprises S. aureus antigen for stimulating the immune response.

10. A method according to claim 7 wherein the immunostimulant stimulates a non-specific mucosal immune response against S. aureus.

11. A method according to claim 10 wherein the immunostimulant is a probiotic.

12. A method according to claim 11 wherein the probiotic consists of one or more strains of bacteria selected from the group consisting of Lactobacillus, Bifidobacterim, Brevibacterium, Propionibacterium Mycobacterium, and mixtures of immunologically active or adjuvanting parts of the foregoing.

13. A method according to claim 12 wherein the probiotic consists of whole viable or killed cells.

14. A method according to claim 12 or 13 wherein the probiotic stimulates a Th1 immune response and/or suppresses a Th2 immune response in the subject.

15. A method for improving sleep quality of a subject, comprising administering to the subject an effective amount of an agent for reducing or inhibiting infection or colonisation of the nasal cavity of the subject by Staphylococcus aureus.

16. A method according to any one of claims 1 to 15 wherein the subject is a human.
